(19) United States
(12) Patent Application Publication
(10) Pub. No.: US 2019/0269886 A1
(43) Pub. Date: Sep. 5, 2019

(54) MEDICAL DEVICE WITH REDUCED OCCLUSION

(71) Applicant: Becton, Dickinson and Company, Franklin Lakes, NJ (US)

(72) Inventors: Gail SIEWIOREK, Winchester, MA (US); Curtis BLANCHARD, Riverton, UT (US); John KNIGHT, Clay Center, NE (US); Yiping MA, Layton, UT (US)

(73) Assignee: Becton, Dickinson and Company, Franklin Lakes, NJ (US)

(21) Appl. No.: 16/345,447

(22) PCT Filed: Oct. 25, 2017
(86) PCT No.: PCT/US2017/058279

§ 371 Date: Apr. 26, 2019

Related U.S. Application Data
(60) Provisional application No. 62/414,959, filed on Oct. 31, 2016.

(51) Int. Cl. A61M 25/00; A61M 5/142; A61M 5/168

(52) U.S. Cl. CPC A61M 25/0068; A61M 2005/1581; A61M 5/16831; A61M 5/142

(57) ABSTRACT

A medical device, such as a catheter, for use in medical applications includes an elongated body (26) with a tip having an opening (32) at one end. The catheter tip is configured to inhibit occluding and to permit outflow of fluid medication from the catheter to ensure proper delivery of the medication to an intended target area. The tip of the catheter is constructed to have at least one beveled end surface (42) to assist insertion into the patient and a radial thickness (49) at a critical bend point substantially equal to the radius (41) of the fluid path (36) and at a location spaced from the end a distance substantially equal to the radius of the fluid path to inhibit folding and occluding the catheter during use.

This application claims priority under 35 U.S.C. § 119(e) from U.S. Provisional Application Ser. No. 62/414,959, filed Oct. 31, 2016, which is incorporated herein by reference in its entirety.

### FIELD OF THE INVENTION

The present invention relates generally to medical devices, such as catheters, used in medical treatment and methods of use thereof The invention is particularly directed to medical devices, such as catheters, that are configured to inhibit occlusion during use and insertion into the patient with a reduced incidence of folding and occlusion.

### BACKGROUND OF THE INVENTION

Delivery devices such as infusion pumps and infusion sets are known for delivering a medication or drug to a patient over a prolonged period time. These devices typically include a soft flexible catheter that is inserted into the patient at a suitable depth for the drug delivery. A rigid cannula is often used as an inserter needle that extends through the catheter for penetrating the skin and positioning the catheter in the patient. After insertion, the cannula or insertion needle can be removed, leaving the catheter in the skin of the patient.

One example of an infusion set is sold under the trademark Quick-Set® infusion set by Medtronic. The infusion set includes a catheter assembly connected to a pump (e.g. MiniMed Paradigm® insulin pump by Medtronic) by a tubing set. A separate insertion device inserts and/or attaches the catheter assembly to a user by an introducer needle provided as a part of the infusion set. The catheter assembly can also be inserted manually into a user's skin. The infusion set and insertion device can also be combined, as in the Mio® infusion set sold by Medtronic that combines the infusion set and insertion device into one unit.

Another example of an insulin infusion device is referred to as a patch pump. A patch pump is an integrated device that combines most or all of the fluid components in a single housing that is adhesively attached to an infusion site, and does not require the use of a single housing that is adhesively attached to an infusion site, and does not require the use of a separate infusion or tubing set. A patch pump adheres to the skin, contains insulin or other medication, and delivers the drug or other substance over a period of time, either transdermal, or via an integrated subcutaneous catheter. Some patch pumps communicate with a separate controller device wirelessly such as one sold under the brand name OmniPod®, while others are completely self-contained. Both conventional pump infusion sets and patch pumps need to be reapplied on a frequent basis, such as every three days, as complications may otherwise occur.

These devices typically include flexible catheters that are inserted into the skin by the introducer needle, as is well known in the art. Once the introducer needle is removed, generally through the catheter, the catheter is enabled to deliver insulin. When the catheter is attached to a user, the catheter can become occluded. The tip of the catheter that dispenses the insulin to the user can become obstructed due to the formation of a blockage, such as tissue inflammation. In addition, the catheter may develop kinking, such that the catheter becomes snagged, knotted, or sharply bent to form a kink that impedes or blocks fluid flow out of the tip of the catheter.

The occlusion can be caused by mechanical problems, such as sliding back in an accordion or bellows fashion or the tip folding back on the introducer needle during insertion. In addition, kinking may also occur during deployment caused by a blunt end on the leading end of the catheter, which may cause excess force to be transmitted to the catheter as the catheter initially penetrates the outer surface of the skin. Similarly, excessive bounce or vibration in the insertion mechanization may also result in excessive force being transmitted to the catheter.

Occlusion can also be caused by biologic or pharmacologic and/or mechanical obstruction of the catheter tip by tissue structures. Depending on the level of irritation caused by the catheter and the movement allowed by the catheter adapter/hub, the tissue can become inflamed as part of a foreign body response, resulting in reduced insulin uptake. Further, there is a tendency for insulin to crystallize when flow is reduced to a minimum (low basal flow) or temporarily stopped, e.g. for bathing, swimming or extended periods, during which time the infusion set is disconnected from the pump. Insulin crystallization that is allowed to proliferate will ultimately occlude the catheter to a point at which the required pump pressure can exceed the normal flow conditions of the pump and trigger an alarm.

The tip of the catheter can also be blocked by an external force to the infusion site that can cause the open end of the catheter to press against tissue structures in the body. This phenomenon has been demonstrated in model tests in which a slight force is applied to the infusion hub in a downward direction, and it can be observed, via fluoroscopy, that the catheter is occluded at the tip.

It is highly desirable, to minimize the risks of occlusion, kinking, and other complications while maintaining a degree of comfort to the user, because once the catheter becomes fully or partially blocked, infusion therapy cannot take place at all, or can be reduced below target flow rates. Soft plastic catheters are prone to kink or occlude with normal wear, while the rigid catheters are often found to be uncomfortable to the user, because the rigid catheter tends to move around within the tissue of the user. Both soft plastic catheters and rigid catheters can also exhibit other undesired complications such as tissue inflammation and foreign body response.

Insulin infusion devices currently available on the market generally incorporate either a flexible catheter made of soft materials, such as soft plastic, fluorinated polymers, Teflon®, and so forth or a rigid catheter, such as a stainless steel cannula. A rigid cannula has a sharp tip, which is used to pierce the skin, similar to an introducer needle in a conventional inserter. Such products are recommended for individuals who have a high incidence of catheter kinking and are not recommended for use beyond two days, because they can occlude for the reasons mentioned above.

Accordingly, a need exists for an improved design, configuration and construction of medical devices to reduce the occurrence of occlusion.

### SUMMARY OF THE INVENTION

The objects of the present invention are to provide medical devices configured and shaped to optimize fluid flow out of the medical device while maintaining column strength for catheter insertion, axial and radial strength for resistance to deformation, flexibility for user comfort, and tensile strength for durability, insertion and removal. The medical device in one embodiment can be a catheter and particularly a soft, flexible catheter such as those used in infusion sets for insulin injection.

These and other objects are substantially achieved by providing a medical device assembly wherein the medical device provides a tip having a shape and dimension that permits proper delivery of a fluid substance, such as insulin doses, to the user while reducing the incidence of kinking and/or occlusion of the device at the open end of the tip. In particular, one feature of the invention is to provide a device having a discharge or dispensing end having a shape and configuration that resists or inhibits the end or tip of the device from bending and folding inwardly into the fluid pathway thereby inhibiting occlusion during use.

In one embodiment, the medical device is an infusion set having a catheter that includes an elongate member having a sidewall, a first end portion, a second end portion, and an opening at each of the end portions, and a fluid pathway or lumen through the elongate member between the openings of the end portions of the elongate member, where a tip has a bevel to assist in insertion of the catheter into the patient and a thickness at a predetermined location from the tip to inhibit occlusion of the tip.

In another embodiment, the medical device is a catheter including an elongate member having a sidewall, a first end and a second end, an opening at each of the end, and a fluid pathway extending through the elongate member between the openings at the end of the elongate member, where the first end has a beveled end portion extending from a tip of the catheter and a tapered portion extending from the beveled end. The beveled end portion extends from the open end at an angle to assist in penetration of the catheter into the patient and where the radial thickness of the end portion at a bend point about equal to the radius of the fluid pathway at the tip is sufficient to inhibit folding or occlusion of the end portion of the catheter at the bend point.

Another embodiment the medical device can be a catheter having an elongate member having a tip with a beveled end at a first angle with respect to the longitudinal axis of the elongate member and a tapered portion extending from the beveled end at a second angle relative to the longitudinal axis and where the second angle is greater than the first angle to form a concave recessed profile of the end of the catheter.

A further embodiment provides a catheter having an elongate member with a rounded convex shaped end portion with a first radius of curvature and tapered portion extending from the rounded end portion with a second radius of curvature that is less than the first radius of curvature.

Another embodiment provides a method of administering a substance by a medical device such as a flexible catheter. The method includes the steps of providing a catheter with an elongate member having a sidewall, a first end, a second end, an opening at each of the ends, a fluid pathway through the elongate member between the openings of the ends of the elongate member, where the first end portion has a beveled portion with a first angle and a tapered portion extending from the beveled portion at a second angle, and where the beveled portion has a thickness to inhibit occluding of the catheter The method further includes inserting the catheter into a patient and administering a substance to the patient through the catheter.

Another embodiment provides an infusion system having a base, a hub detachably attached to the base, and a pump. The system includes a fluid tubing set that connects the pump and the base and a catheter, cannula, needle or other medical device with a fluid pathway through an elongate member.

The features of the invention are basically attained by providing a medical device comprising an elongate member comprising a sidewall, a first end portion with a first open end, second end portion with a second open end, a fluid pathway extending through the elongate member between the first open end and second open end. The first end portion has an inner surface forming the fluid pathway and an outer surface. The outer surface has a first beveled portion converging from the first open end, and where the sidewall at the first end portion has a first radial thickness at a location spaced from said first open end substantially equal to a radius of the fluid pathway to inhibit occlusion of said the open end during use.

The features of the invention are further attained by providing a medical device comprising: an elongate member comprising a sidewall, a first end portion, a second end portion, and an opening at each of the end portions; a fluid pathway extending through the elongate member between the openings at the end portions of the elongate member; and where the first end portion has a beveled end portion with a critical bend portion located a distance from the opening of the first end portion substantially equal to a radius of the fluid pathway, and where the elongate member has a radial thickness at the critical bend portion sufficient to resist folding of the end portion during insertion and use of the device.

Additional and/or other aspects and advantages of the present invention will be set forth in the description that follows, or will be apparent from the description, or may be learned by practice of the invention.

### BRIEF DESCRIPTION OF THE DRAWINGS

The various objects, advantages and novel features of the exemplary embodiments of the present invention will be more readily appreciated from the following detailed description when read in conjunction with the appended drawings, in which:

FIG. 1 is a perspective view of a infusion set including the medical device in the form of a catheter of the invention;

FIG. 2 is an enlarged cross-sectional view of an end portion of the intravenous medical device shown as a catheter assembly of FIG. 1;

FIG. 3 is a front view of a medical device in accordance in an embodiment of the present invention;

FIG. 4 is side view of the medical device of FIG. 3 showing the fluid pathway in phantom lines;

FIG. 5 is a partial cross-sectional view of the medical device tip of FIG. 3 showing the angles and dimensions;

FIG. 6 is an enlarged partial cross-sectional view of the medical device of FIG. 3;

FIG. 7 is a side view of a catheter in a second embodiment of the invention;

FIG. 8 is an enlarged side view of the tip of the catheter of FIG. 7 showing the angles and dimensions of the tip and end portion;

FIG. 9 is partial cross-sectional side view of a catheter end portion in a third embodiment;

FIG. 10 is a partial cross-sectional side view of the catheter end portion in a fourth embodiment;

FIG. 11 is partial cross-sectional side view of a catheter end portion in a fifth embodiment;

FIG. 12 is side view of a catheter end portion in a sixth embodiment showing a rounded, convex shaped tip;

FIG. 13 is side view of a catheter tip in a seventh embodiment showing a rounded tip surface;

FIG. 14 is partial cross-sectional side view of a catheter end portion in an eight embodiment;

FIG. 15 is a side view of the catheter of FIG. 14; and

FIG. 16 is a partial cross-sectional side view of a catheter end portion in a ninth embodiment.

### DETAILED DESCRIPTION OF THE EXEMPLARY EMBODIMENTS

Reference will now be made in detail to embodiments of the present invention, which are illustrated in the accompanying drawings, wherein like reference numerals refer to like elements throughout. The embodiments described herein exemplify, but do not limit, the present invention by referring to the drawings. As will be understood by one skilled in the art, terms such as up, down, bottom, and top are relative, and are employed to aid illustration, but are not limiting.

The exemplary embodiments described below provide improved medical devices, such as catheters, for use with infusion sets and/or patch pumps, or as intravenous or peripheral catheters. The medical device of the invention is a device that can be inserted and positioned in the patient subcutaneously or intravenously. The medical device can be a probe, cannula, needle, catheter, and the like. The catheter can be a peripheral catheter or an intravenous catheter. In the embodiments described the medical device is a catheter for purposed of illustration but is not intended to be limiting. The medical device can be a hollow, tubular or elongated member having fluid passage or lumen capable for delivering a substance to a patient. The medical device is typically a flexible member, such as a flexible catheter. The catheter can have diameter and width suitable for use in an infusion set for delivering a substance, such as insulin.

In the embodiments shown, the medical device is a soft, flexible catheter. The invention reduces the catheter kinking, occlusion and other undesirable complications, such as tissue inflammation and foreign body response that may act to block or reduce the flow of medication fluids out of the catheter to the patient. The exemplary embodiments are presented in separate descriptions, although the individual features and construction of these embodiments can be combined in any number of ways to meet the therapeutic needs of the user.

It will be understood by one skilled in the art that this disclosure is not limited in its application to the details of construction and the arrangement of components set forth in the following description or illustrated in the drawings. The embodiments herein are capable o of being modified, practiced or carried out in various ways. Also, it will be understood that the phraseology arid terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not limited to physical or mechanical connections or couplings. Further, terms such as up, down, bottom, and top are relative, and are employed to aid illustration, but are not limiting.

In the embodiment shown, the medical device is a catheter generally made of a flexible plastic material, and provides a high level of comfort to the user. The flexible catheters can deliver insulin or other medicaments to the target tissue or area with a reduced incidence of occlusion.

The catheter has a first end forming a tip that is configured to reduce the incidence of folding or collapsing of the tip that can cause occlusion of the catheter while maintaining a shape for ease and comfort of insertion into the skin of the patient without kinking and with reduced incidence of the tip portion folding or occluding during insertion and during use. The catheter is typically configured for use in a delivery device for delivering a drug or other medication, such as insulin to a patient.

The catheters of the invention provide a geometry, shape and configuration to provide a balance between the ease and comfort of insertion into the patient and strength to reduce the incidence of occlusion forming during insertion and during use generally caused by the end or tip of the catheter folding or deflecting inward into the fluid pathway that will restrict the dispensing of a substance. Catheters with a large bevel angle forming a large penetration angle are shown to exhibit high penetration forces during insertion while exhibiting higher strength and resistance to occlusion during use. Catheters having a tip with a lower penetration angle provide easier penetration while exhibiting a higher incidence of occlusion during use. The catheter of the invention can have various dimensions. In one embodiment, the catheter can be a 24 G catheter. In other embodiments, the catheter can be as 26 G or a 28 G catheter. The catheters can have a range of 24-28 G depending on the intended use.

One feature of the invention is to provide a medical device, such as a catheter, that has a tip for insertion into the patient where the tip has a beveled insertion end with a frustoconical angled surface. The angled surface of the tip is oriented with respect to a longitudinal axis that enables ease of insertion while reducing the occurrence of occlusion or tip collapse during use. The risk of catheter tip collapse is higher for small diameter (large gauge) catheters. The catheter is configured to avoid a blunt end that can inhibit proper insertion. The catheters are desirably small diameter, such as for example 26-28 gauge. The tip of the catheter can be configured to provide a 28 gauge flexible catheter provides a suitable time period before the tip folds over, collapses and occludes the tip during use.

The point at which the tip of the catheter folds and collapses is referred to as the critical bend point. The critical bend point is located axially from the distal end of the catheter a distance of one catheter inner radius magnitude. The minimum distance from the tip that can fold over and occlude the catheter is equivalent to the catheter inner radius. It has been found that the thickness of the wall of the catheter can facilitate the structural integrity of the catheter tip and the ability to insert the catheter tip by streamlining the outer profile. Generally a smaller thickness at the critical bend point provides easier insertion but result in collapse and occlusion in a short period of time.

In one embodiment, the critical bend point can have a radial thickness of about 0.002 inch for a 28 gauge catheter. In an embodiment the critical bend point has a thickness of about 0.0029 to 0.004 inch for a 28 gauge catheter. In another embodiment, the critical bend thickness can be about 0.0030 to 0.0035 inch for a 28 gauge catheter.

The catheter tip is configured to provide an outer profile that provides lower penetration forces and increased insertion success while providing a thickness to reduce the incidence of collapse. In one embodiment, the outer profile has a gradual transition between the surface of the tip to reduce the potential for the skin tissue of the patient to interfere with eh insertion into the patient, The entry tip angle provides a thickness to reduce folding at the tip with gradual increased thickness at the critical bend point. Past the critical bend point, a smooth transition is provide to the body and tapered surface of the catheter.

In one embodiment of the catheter, the tip of the catheter is a 28 gauge catheter and includes a first bevel having a bevel angle of not more than 30 degrees to the longitudinal axis. In one embodiment, the bevel can at an angle of about 24 to 28 degrees. In other embodiments the bevel can has a bevel angle of about 26 to 28 degrees. The bevel angle can have a longitudinal length to provide a bevel height or thickness at the proximal end of the bevel of about 0.0034 to about 0.0004 inch. In one embodiment, the bevel height is about 0.0035 to about 0.004 inch for a 28 gauge catheter. In another embodiment, the bevel height is about 0.0038 inch and has at a bevel angle of about 26-30 degrees to provide a smooth insertion and reduced incidence of occlusion at the critical bend point. A tapered surface forms a smooth transition between the proximal end of the bevel and the tapered surface of the catheter. The tapered surface can have a taper angle of about 3-4 degrees and typically about 3.25 to 4 degrees. In one embodiment, the tapered surface has a taper angle of about 3 degrees.

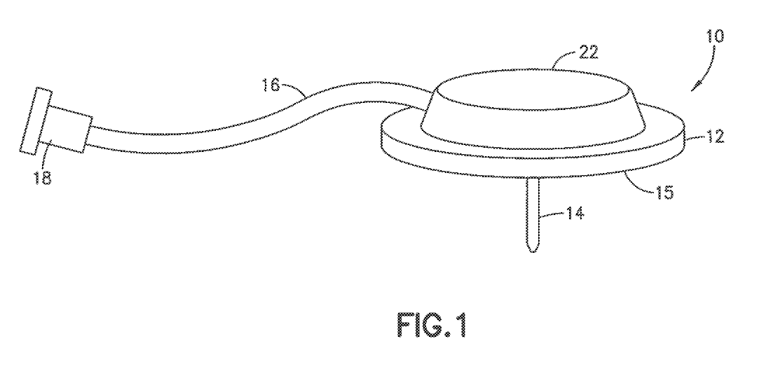

One example of the medical device is a peripheral or intravenous catheter assembly and infusion set 10 as illustrated in FIG. 1. The infusion set is intended to be exemplary of the invention and the medical device. The invention is not intended to be limited to an infusion set or a catheter for use with an infusion set. The infusion set 10 includes a hub or fluid connector 22 that detachably connects with a base 12, a fluid tubing set 16 and a connector 18 that attaches to a pump. The infusion set 10, which includes the hub 22 and the fluid tubing set 16, is attached to or detached from the base 12. The base 12 includes an infusion adapter for connecting with the fluid connector or hub 22. An adhesive pad 15 is attached to the base 12 to secure the base to a user's skin. A catheter 24 is attached to the base 12. Catheters for infusion sets, for example, subcutaneous or intradermal target layers of the skin are generally shorter than intravenous catheters.

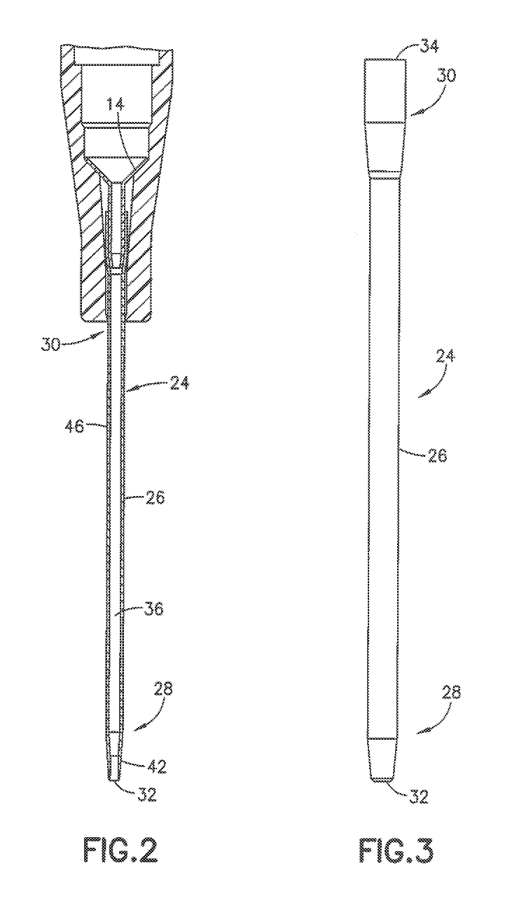

Referring to FIG. 2, the infusion set assembly 10 includes a wedge 14, usually made of a hard substance such as metal or a rigid plastic and having a funnel shape, to which an end portion of the catheter 24 is frictionally attached to connect the catheter to the wedge 14 and a catheter hub. The wedge 14, to which the catheter 24 is attached, is secured to the hub or adapter to form the catheter assembly 10. Fluid exits the tip of the catheter 24 for delivery to the patient.

FIG. 3 shows a side view of the catheter in one embodiment of the medical device. As shown, catheter 24 has an elongate member 26 with a first end portion 28 and a second end portion 30. First end portion 28 has a first open end 30 forming a tip and second end portion 30 has a second open end 32 defining a fluid pathway 36 extending between the first open end 30 and the second open end 32. The catheter in various embodiments of the invention can be 24 Gauge depending on the intended use. In other embodiments, the catheter can be 26 Gauge or 28 Gauge. The catheter 24 is typically used in conjunction with an insertion needle or cannula as known in the art. For simplicity and ease of illustration of the invention, the insertion needle is not shown in the drawings illustrating the details of the catheter.

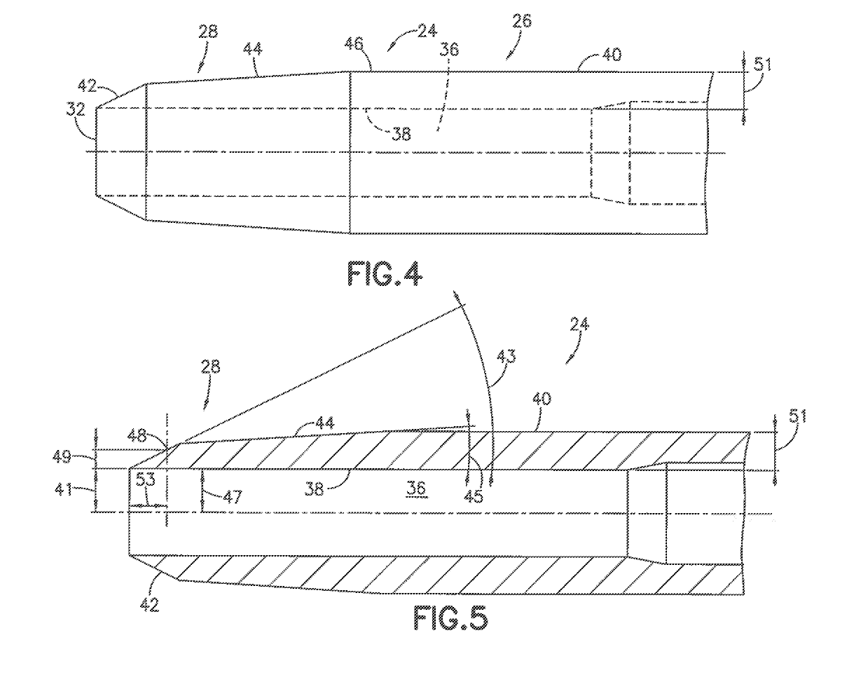

Referring to FIGS. 4 and 5, catheter 24 has a length and diameter suitable for insertion into the patient to deliver the drug, medicament, or other substance while providing comfort to the user. As discussed above, the catheter of this embodiment and the other embodiments disclosed herein are generally positioned in the patient by use of an insertion needle that is later removed for delivering the substance, such as insulin to the patient. The catheter 24 can have a length of about 6-9 mm but can be up to 17 mm in certain embodiments depending on the intended use. The diameter is defined by the gauge as previously discussed. The first end portion 28 has a shape and dimension for inserting into the patient while maintaining the shape and configuration to inhibit the tip end from folding over to close the open end of the catheter and restricting fluid flow. Catheter 24 had an inner surface 38 forming the fluid pathway 36 and an outer surface 40. As shown, the first end portion 28 includes a beveled surface 42 extending from the first open end 32 toward the second end 30 at an inclined angle with respect to the longitudinal axis of the catheter and the fluid pathway 36. In the embodiments shown in the drawings, the dimensions are primarily in relation to a 28 G needle. The dimensions and angles can be modified as needed for other needle gauges. The dimensions and angles in the drawings and as described are for illustrative purposes and are not intended to limit the scope of the invention.

The catheter 24 has a point or location defined as a critical bend point that corresponds substantially to a point or location on the catheter that is spaced from the first open end 32 a distance corresponding substantially to the inner radius of the fluid pathway 36. The critical bend point is a location where the catheter is prone to bending inwardly toward the center of the pathway 36 resulting in occlusion or restriction of the pathway. The occlusion can be partial where one or more portions can fold inwardly to restrict the outlet of the catheter. To inhibit occlusion by the end portion folding or bending inwardly toward the center of the pathway, the invention is directed to providing a radial thickness of the catheter at the critical bend point that is sufficient to inhibit occlusion while providing a beveled angle that provides the comfort during insertion and during use of the catheter by the patient. The shape and outer dimension of the first end portion 28 are configured to provide case of insertion force into the patient while preventing or inhibiting occlusion.

As shown in FIG. 4, first end portion 28 of catheter 24 has a tapered portion 44 and a substantially cylindrical body portion 46. The tapered portion 44 extends between the distal end of the beveled surface portion 42 to the cylindrical body potion 46 forming a substantially frustoconical shaped end. The beveled portion 42 and the tapered portion 44 are configured to provide comfort during insertion and during use while inhibiting occlusion during use of the catheter by providing a thickness at the critical bend point to resist folding inward. The angle of the beveled portion 42 forms a leading edge at the first open end and extends away from the first open end to enable insertion into the skin of the patient. In the embodiment shown, the angle of the beveled portion 42 relative to the longitudinal axis is greater than the angle of the tapered portion 44. In this embodiment, the angle 43 of the beveled portion 42 is about 30 degrees and the angle 45 of the tapered portion 44 is about 4 degrees relative to the longitudinal center axis of the catheter and pathway 36. In this embodiment, the first beveled surface is at an angle greater than the angle of the second beveled surface which forms a substantially convex outer surface at the end of the device. In other embodiments, the first bevel can be at an angle less than the angle of the second beveled surface which can form a substantially concave outer surface.

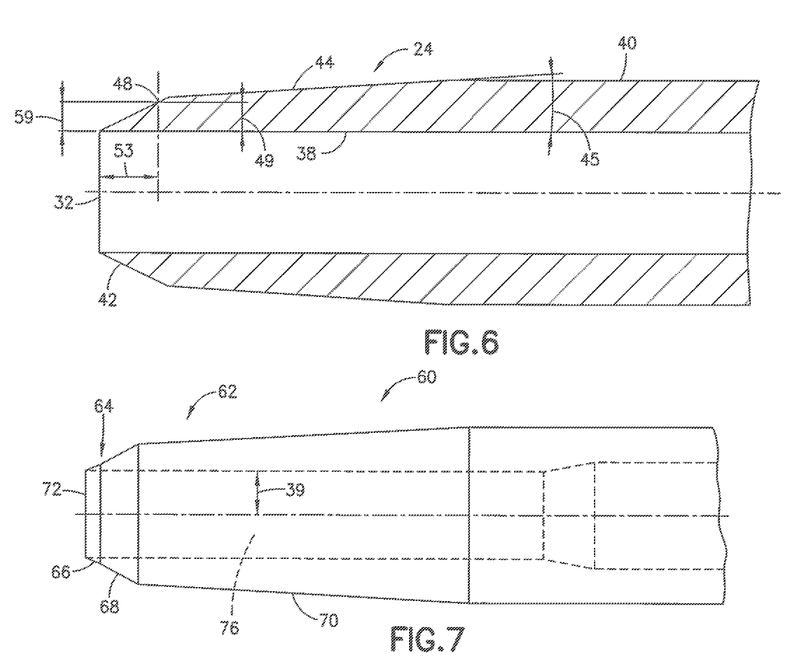

As shown in FIGS. 5 and 6, the critical bending point 48 is on the beveled portion 42 and defined as the point spaced from the first open 32 a distance corresponding to the radius 47 of the pathway 36. In the embodiment shown, the dimensions are intended to exemplary without limiting the invention to the specific dimensions. In this embodiment, the critical bending point is positioned in the surface of the beveled portion 42 and has a radial thickness to inhibit bending during use. In the embodiment of FIGS. 4-6, the catheter can be 28 G having cylindrical body portion 46 with a thickness 51 of about 0.0057 inch and the fluid pathway 36 has a radius 47 of about 0.0056 inch so that the critical bend point is spaced from the open end about 0.0056 inch as indicated by reference 53. The beveled surface 42 in the embodiment shown in FIGS. 5 and 6 can have a fold over thickness 55 of about 0.00323 inch and a fold over length 53 of 0.0056 inch. In the embodiment shown, the bevel 42 has a longitudinal length 57 of about 0.0066 inch and a bevel height 59 of a about 0.0038. The critical bending point 48 has radial thickness 49 of about 0.00323 inch. In one embodiment, the catheter can have a radial thickness at the critical bending point of at least 0.003 and less than 0.006 inch. In another embodiment, the catheter can have radial thickness at the critical bend point 48 of about 0.003 to 0.004 inch.

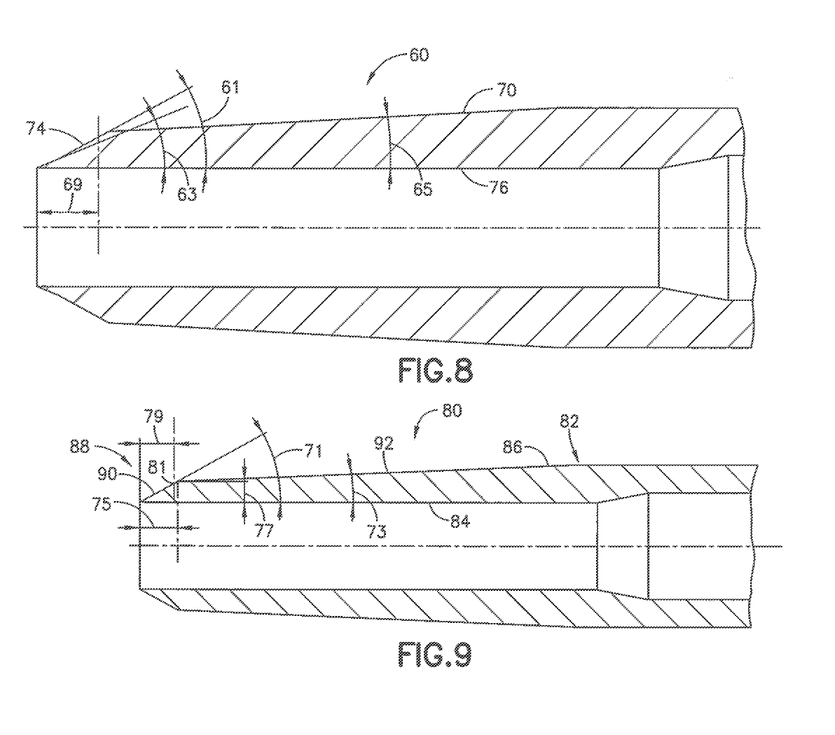

In a second embodiment shown in FIGS. 7 and 8, a catheter 60 has a similar shape as in the embodiment of FIGS. 4-6 except for the first end portion 62. As in the previous embodiment, the catheter 60 can be incorporated into the infusion set 10. For purposes of illustration, the infusion set is not shown in the figures. The first end portion 62 has a beveled portion 64 defined by a first bevel 66 and a second bevel 68. The first bevel 66 extends from the distal end of the first portion 62 to the second bevel 68. The second bevel extends from the end of the first bevel 66 to a tapered surface portion 70. In this embodiment, the first bevel 66 extends at an angle that is less than the angle of the second bevel 68 relative to the longitudinal axis of the catheter. The different angles of the first bevel 66 and second bevel 68 forms an annular recess in the outer surface of the beveled portion 64. The low angle of the first bevel 66 provides a smooth insertion of the catheter into the patient compared to a steep angle that forms a comparatively blunt end that contacts the skin during insertion. The steeper angle of the second bevel 68 enables a thickness of the first end portion to be attained to resist folding and occlusion of the first open end 72 of the catheter during use and insertion. In other embodiments, the first bevel can be at an angle less than the angle of the second bevel.

In this embodiment, the critical bending point 74 shown in FIG. 8 corresponds to the distance from the first open end 72 substantially equal to the radius of the fluid pathway 76. As shown in FIG. 8, critical bending point 72 is located in the second bevel 68 and positioned close to the tapered portion 70. In the embodiment shown, the first bevel 66 is oriented at an angle 61 of about 24 degrees and the second bevel 68 is oriented at an angle 63 of about 30 degrees relative to the longitudinal axis of the catheter and pathway 76. The tapered portion is angled at about 3 degrees relative to the longitudinal axis indicated by reference 65. The catheter can be 28 G having a cylindrical body portion 78 of the catheter with a thickness of about 0.0057 inch and the critical bending point has a thickness of about 0.00323 inch at a position spaced about 0.00575 inch form he end 72 indicated by reference 69. In the embodiment shown, the first bevel 66 can have a longitudinal length 67 of about 0.0020 inch. The combined longitudinal length of the first bevel 66 and second bevel 68 can be about 0.00575 inch.

FIG. 9 illustrates an end portion and a tip of a catheter in another embodiment of the invention. The catheter 80 of FIG. 9 can also be used in the infusion set 10 of FIG. 1. The catheter 80 is similar to the previous embodiment having an elongate body member 82 with a fluid pathway defining an inner surface 84 and an outer surface 86. A first distal end 88 of catheter 80 includes a bevel 90 oriented at an inclined angle 71 of about 30 degrees relative to the longitudinal axis of the fluid pathway and catheter 80. A tapered portion 92 extends from the edge of the bevel 90 at an inclined angle 73 of about 2.5 degrees. In this embodiment, the critical bend point is about 0.0056 inch from the distal end of catheter 90 indicated at 75, a bevel height 77 of 0.00310 inch and a bevel length 79 of 0.0054 inch. In the embodiment shown, the bevel length corresponds substantially to the critical bend point so that the thickness of the catheter at the critical bend point 81 is about 0.00310 inch.

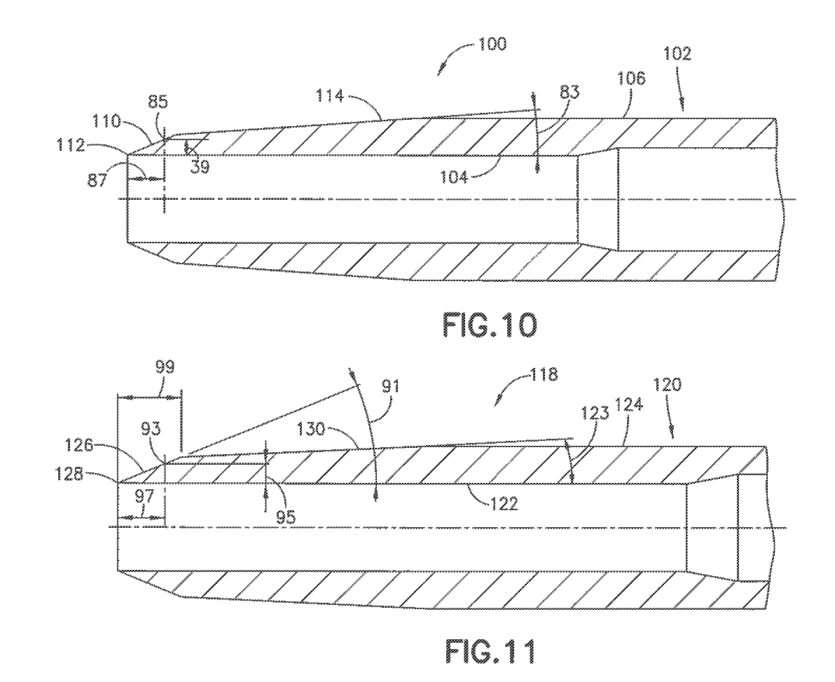

Referring to FIG. 10, a further embodiment of the invention is illustrated where the catheter 100 includes an elongated body member 102 with an inner surface 104 and outer surface 106. The catheter 100 can be used with the infusion set 10 of FIG. 1. A distal first end 108 includes a bevel 110 extending at an inclined angle of about 22.4 degrees from the tip 112 of the catheter relative to a longitudinal axis of catheter 100 and the fluid pathway. A tapered surface 114 extends from the edge of the bevel 110 at an angle 83 of about 4 degrees relative to the longitudinal axis of the catheter 100. The bevel angle forms a critical bend point 85 at a distance 87 of about 0.00560 inch from the tip 112 and a thickness 89 at the critical bend point of about 0.00231 defining a fold over length. In this embodiment, the critical bend point is oriented in the bevel 110.

FIG. 11 shows another embodiment of the invention where the catheter 118 having an elongated body member 120 with an inner surface 122 and an outer surface 122. As in the previous embodiments, the catheter 118 can be used with the infusion set 10. A first distal end 124 includes a bevel 126 extending from a tip 128 at an inclined angle of about 22 degrees. A tapered surface 130 extends from the end of the bevel 126 at an angle 91 of about 2.5 degrees relative to the longitudinal axis of catheter 118 and the fluid pathway. The catheter cart be, for example, 28 G. The bevel angle 91 forms a critical bend point 93 with a thickness of about 0.00226 inch at about 0.0056 inch from tip 128 indicated by reference 97. As in the previous embodiment, the critical bend point is oriented in the bevel 126. In this embodiment, bevel 126 has a longitudinal length 99 of about 0.0099 inch.

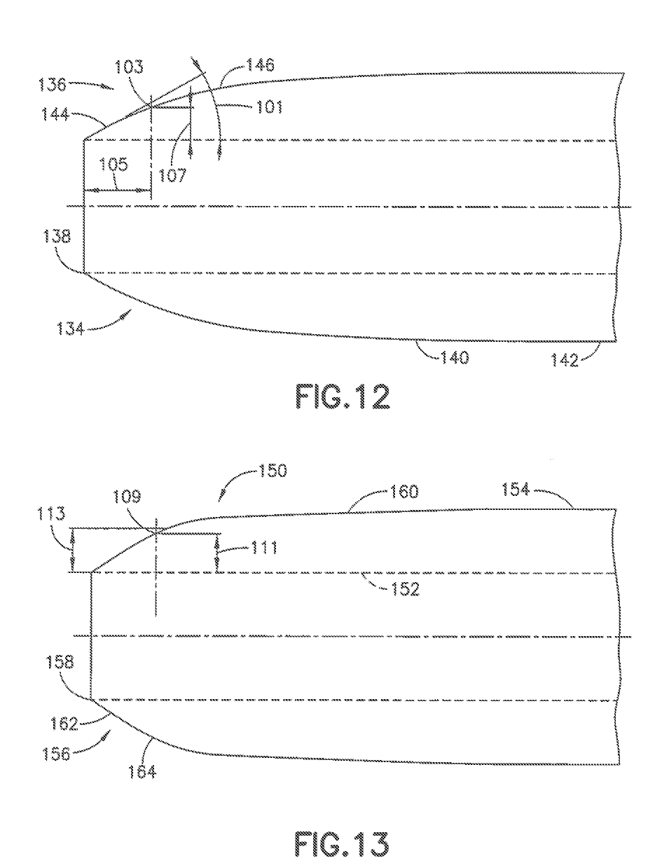

FIG. 12 shows another embodiment of the invention where the catheter 134 for use in the infusion set 10 has a first end portion 136 with a substantially continuous rounded outer face that converges to a tip 138. A tapered surface 140 extends from the edge of the first end portion 136 toward a second end 142 as in the previous embodiments. In this embodiment, first end portion 136 includes a first bevel end portion 144 extending from the tip 138 at angle 101 of about 30 degrees forming an initial penetration angle and transitions to a smaller angle toward the tapered surface 140. First bevel 144 merges with a second bevel end portion 146 forming a smooth, continuous surface extending between tip 138 and tapered surface 140. In this embodiment, the angle of the second bevel portion is smaller than the angle of the first bevel portion. In a similar manner the radius of curvature of the first bevel can be greater than the radius of curvature of the second bevel. The critical bend point 103 is spaced from tip 138 a distance 105 of about 0.0056 inch and has a thickness 107 at the critical bend point of about 0.0029 inch. The continuous rounded surface of first end portion 136 provides a smooth transition between the first bevel 144 and the second bevel 146 and with the tapered surface 140, In this embodiment, the critical bend point has a thickness 107 of about 0.0029 inch.

FIG. 13 shows a further embodiment of the invention of a catheter 150 having an inner surface 152 and outer surface 154. The catheter can be, for example, 28 G. A first end portion 156 extends from a tip 158 to a tapered surface 160. The first end portion 156 has a continuously curved substantially convex shape to form a rounded smooth surface extending between tip 158 and tapered surface 160. The first end potion 156 has a first bevel 162 forming an initial penetration angle of about 35 degrees. The first bevel 162 curves to a second bevel 164 at a smaller angle to converge with the tapered surface 160. In this embodiment, the critical bend point 109 has a thickness of about 0.00353 inch indicated by reference 111. The first bevel 162 has a radial thickness 113 of about 0.0038 inch.

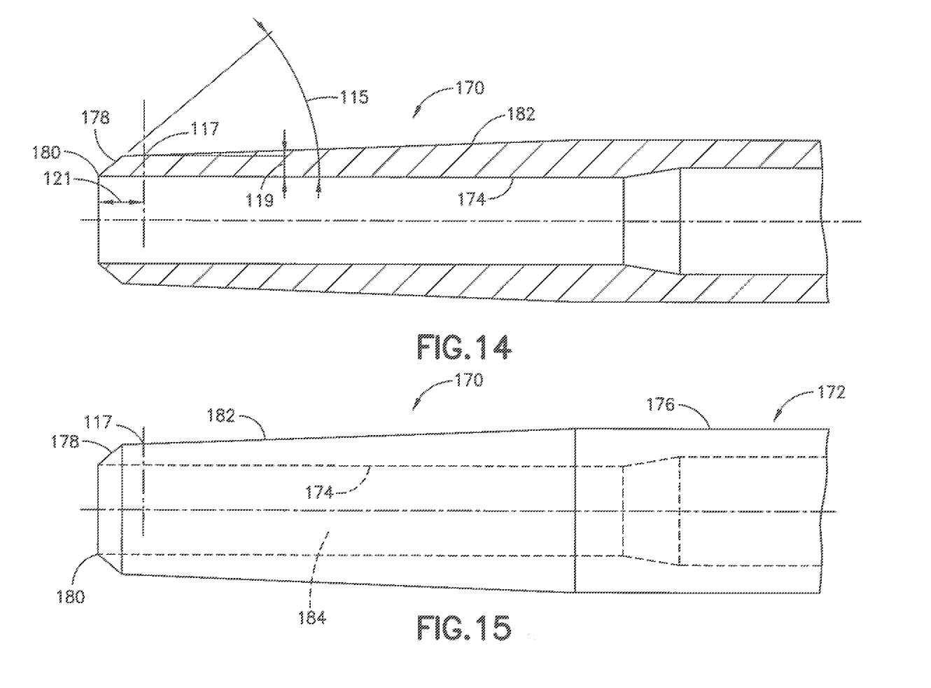

FIGS. 14 and 15 illustrate another embodiment of the invention of a catheter 170 similar to the previous embodiments for use in the infusion set 10 where catheter 170 has an elongated body member 172 with an inner surface 174 and outer surface 176. A first end potion defined by a bevel 178 extends from a tip 180 to a tapered surface 182. In this embodiment, bevel 178 faints a substantially conical shaped surface forming a penetration surface for the catheter. The bevel 178 has an angle 115 of about 38.7 degrees relative to the longitudinal axis of catheter 170 and the fluid pathway 184. In this embodiment, the critical bend point 117 has a thickness of about 0.0028 inch corresponding to a fold over height and an axial length 119 or spacing from the tip 180 of about 0.0056 inch. The bevel 178 can have a longitudinal length 12 of about 0.0035 inch.

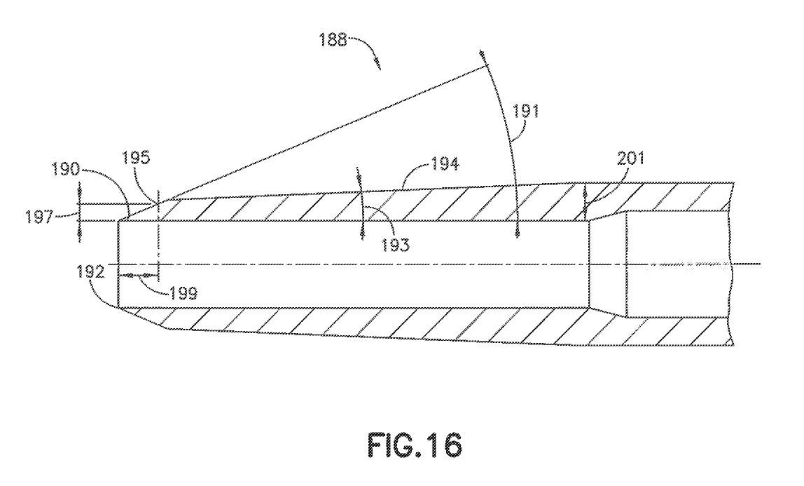

FIG. 16 shows a catheter 188 in another embodiment similar to the embodiment of FIG. 14 and FIG. 15 where the catheter can used in the infusion set 10. In this embodiment, catheter has a first end portion defined by a bevel 190 extending at an angle 191 from a tip 192 of about 22.40 degrees. Bevel 190 extends from tip 192 to a tapered surface 194 oriented at an incline angle 193 of about 2.50 degrees. The critical bend point 195 has a thickness 197 or a radial dimension of about 0.00231 inch. The critical bend point 195 can have a fold over length 199 of about 0.0056 inch. The bevel 190 can have longitudinal length of 0.0075 inch. The catheter can have a thickness 201 at the end of the tapered portion of about 0.0057 inch.

In a preclinical study, swine were placed under anesthesia and the catheters of the invention were introduced for three days to determine the occlusion based on the shape and dimensions of the first end portion and tip of the catheters. The results showed a relationship between the penetration angle and the insertion force necessary for insertion and a relationship between the thickness at the critical bend point and the occlusion after three days. The tests showed that the catheter of FIGS. 6 and 7 having a bevel angle of 30 degrees and a critical bend thickness of 0.0056 inch exhibit minimal occlusion after three days. The embodiments having a smaller bevel angle forming the insertion angle resulted in better initial penetration into the patient but a higher incidence of occlusion after three days. For example, the embodiment of FIG. 10 having a bevel angle of 22.4 degrees showed a greater ease of insertion with a greater incidence of occlusion compared to the embodiment of FIGS. 4-6 and the embodiment of FIGS. 7 and 8.

Although only a limited number of exemplary embodiments of the present invention have been described in detail above, those skilled in the art will readily appreciate that many modifications are possible in the exemplary embodiments without materially departing from the novel teachings and advantages of this invention. It is particularly noted that the features of different embodiments and claims may be combined with each other as long as they do not contradict each other. Accordingly, all such modifications are intended to be included within the scope of this invention as defined in the appended claims and their equivalents.

What is claimed is:

1. A medical device comprising:
an elongate member comprising a sidewall, a first end portion with a first open end, second end portion with a second open end;
a fluid pathway extending through the elongate member between the first open end and second open end; and
said first end portion having an inner surface forming the fluid pathway and an outer surface;
said outer surface having a first beveled portion converging from said first open end, and where said sidewall at said first end portion has a first radial thickness at a location spaced from said first open end substantially equal to a radius of said fluid pathway to inhibit occlusion of said first open end during use.

2. The medical device as claimed in claim 1, wherein said medical device is a flexible catheter having a critical bend point at said location spaced from said first open end a distance substantially equal to a radius of the fluid pathway at said first open end.

3. The catheter as claimed in claim 2, wherein the critical bend point is positioned along said first beveled portion.

4. The medical device as claimed in claim 2, wherein the critical bend point is spaced between said first beveled portion and said second open end.

5. The medical device as claimed in claim 2, wherein said first beveled portion is at a inclined angle of about 30 degrees relative to a longitudinal axis of said elongate member and where said critical bend point has a radial thickness of about 0.003 inch.

6. The medical device as claimed in claim 2, wherein said critical bend point has a radial thickness to resist the end of said catheter from folding and occluding during insertion and during use.

7. The medical device as claimed in claim 1, wherein said catheter is a between 24 gauge and 28 gauge.

8. The medical device as claimed in claim 2, wherein said first beveled portion is at a first angle and said first end portion includes a second beveled portion extending from said first beveled portion at a second angle that is different that said first angle, and said elongate member having a tapered portion extending from said second beveled portion at a third angle that is different from said first and second angle.

9. The medical device as claimed in claim 2, wherein said first beveled portion is at an angle greater than said angle of said second beveled portion with respect to the longitudinal axis of said elongate member to define a convex profile of said first end portion.

10. The medical device as claimed in claim 9, wherein the first end portion defines a continuous curve between said first open end and said tapered portion.

11. The medical device as claimed in claim 2, wherein said first beveled portion is at an angle of about 30 degrees and said first end portion further includes a tapered portion extending from said first beveled portion at an angle of about 4 degrees relative to the longitudinal axis of said elongate member.

12. The medical device as claimed in claim 2, wherein said first beveled portion is at an angle of about 24 degrees and said first end portion has a second beveled portion extending from said first beveled portion at an angle of about 30 degrees and a tapered portion extending from said second beveled portion at an angle of about 3 degrees relative to the longitudinal axis of said elongate member.

13. An infusion set comprising:
a catheter having an elongate member including a sidewall, a first end portion, a second end portion, and an opening at said first and second end portions;
a fluid pathway extending through the elongate member between the openings at the end portions of the elongate member; and
where said first end portion has a first beveled end portion on an outer surface of said longitudinal member with a critical bend point located a distance from said opening of said first end portion substantially equal to a radius of said fluid pathway, and where said elongate member has a radial thickness at said critical bend portion substantially equal to the radius of the flexible pathway to resist folding of said end portion during insertion and use of said catheter.

14. The infusion set catheter as claimed in claim 13, wherein the critical bend point is positioned along said first beveled portion.

15. The infusion set as claimed in claim 14, wherein said first beveled portion is at a inclined angle of about 30 degrees relative to a longitudinal axis of said elongate member and where said critical bend point has a radial thickness of about 0.003 inch.

16. The infusion set as claimed in claim 14, wherein said catheter is a between 24 gauge and 28 gauge.

17. The infusion set as claimed in claim 14, wherein said first beveled portion is at a first angle and said first end portion includes a second beveled portion extending from said first beveled portion at a second angle that is different that said first angle, and said elongate member having a tapered portion extending from said second beveled portion at a third angle that is different from said first and second angle.

18. The infusion set as claimed in claim 17, wherein said first beveled portion is at an angle greater than said angle of said second beveled portion with respect to the longitudinal axis of said elongate member to define a convex profile of said first end portion.

19. The infusion set as claimed in claim 17, wherein the first end portion defines a continuous curve between said first open end and said tapered portion.

20. A method of administering a substance to a patient by a medical device, comprising
introducing a catheter into the patient, said catheter having a elongate member with a side wall, a first end, a second end, an opening at each of the end, a fluid pathway through the elongate member extending between the first end and second end, the first end having a beveled portion with a first angle, and a tapered portion extending from the beveled portion at a second angle, and where the beveled portion has a thickness to inhibit folding and occluding of the catheter during use; and
introducing the substance through the catheter to the patient.

21. The method of claim 20, wherein said catheter has a thickness at a distance from said first end substantially equal to a radius of the fluid pathway defining a critical bend point, and where said critical bend point is spaced from said first end a distance corresponding substantially to the radius of the fluid pathway.
